(19) United States
(12) Patent Application Publication
(10) Pub. No.: US 2006/0235446 A1
(43) Pub. Date: Oct. 19, 2006

(54) Article, system, and method for securing medical device to tissue or organ

(76) Inventor: Norman Godin, Geneva (CH)

Correspondence Address: COZEN O'CONNOR, P.C., 1900 MARKET STREET, PHILADELPHIA, PA, 19103-3508, US

(21) Appl. No.: 11/215,904

(22) Filed: Aug. 31, 2005

Related U.S. Application Data
(60) Provisional application No. 60/672,135, filed on Apr. 14, 2005.

(51) Int. Cl. A61B 17/08

(52) U.S. Cl. 606/151

(57) ABSTRACT

An surgical tilt-tag staple comprising an elongated joining member having a first end and a second end, a first tilt-tag joined to the first end of the joining member and a second tilt-tag joined to the second end of the joining member, each of the first and second tilt-tags normally aligned perpendicularly to the joining member, the tilt-tags bendable under resistance into a position substantially parallel to the joining member and returning to the normal position when resistance is removed, is used to attach a medical device to the inner mucosa of a gastro-intestinal tract of a patient using ultrasound real time visualization and a flexible endoscope. Tilt-tag staples constructed of nickel-titanium alloy are preferred. Removal of such tilt-tag staples can be facilitated by cooling a tilt-tag. Medical devices with an electrical lead can be implanted without conventional suturing by using one terminal tilt tag on the lead to implant it.

### CROSS-REFERENCE TO RELATED APPLICATIONS

Benefit of provisional application No. 60/672,135 filed Apr. 14, 2005 is claimed, the contents of which are incorporated by reference.

### BACKGROUND OF THE INVENTION

The present invention relates to the field of medicine, particularly to suturing and securing medical devices to tissue or organs.

The most common way to secure medical tissues to tissues or organs is by stapling with conventional surgical staples formed from a single length of wire in an approximate U-shape. For example, McGarry, et al., in U.S. Pat. No. 5,366,479, disclose endoscopic application of staples for attaching surgical mesh to body tissue in laparoscopic hernia surgery. In this patent, the stapler is a rigid instrument and cannot be used through the mouth into the esophagus where a flexible instrument is needed.

For certain types of devices placed through the mouth, conventional stapling with a rigid stapler is not possible and so various alternatives using flexible instruments have been devised. For example, to treat a condition known as Gastro-Esophageal Reflux Disease (GERD), a tubular valve has been designed for letting free passage of food from the esophagus into the stomach but stopping food and gastric content such as hydrochloric acid and bile from refluxing from the stomach into the esophagus. Godin, U.S. Pat. No. 5,861,036, described a Gastro-esophageal Anti-Reflux Device (GARD) and Godin, U.S. Pat. No. 6,764,518 described a system for securing the GARD comprising a ring which could be collapsed into a smaller diameter for placement through the mouth and placed in a hiatus hernia after calibration of the diameter of the hernia with a catheter. Such ring was designed to place the GARD tubular valve in the lower esophagus or in a hiatus hernia associated with severe GERD and keep it in place for a significant amount of time, such as months and years, as severe GERD is a chronic condition.

While the aforementioned GARD ring device and method worked for some patients, it was insufficient in certain cases.

In a different approach to addressing GERD, endoluminal fundoplication surgery, a flexible endoscope was disclosed by Adams, et al., in U.S. Pat. No. 6,736,828, as being useful in endoluminal fundoplication surgery where a bonding agent is injected into tissue which forms an intussusception formed by pulling a selected portion of the esophagus into the stomach and displacing a fundus portion of the stomach towards the esophagus, placing a fastener across the intussusception for maintaining an esophageal wall and a gastric wall forming the intussusception adjacent to one another and then injecting the bonding agent to bond the intussusception. Adams, et al., did not describe a flexible echo endoscopic procedure. The intussusception site is located by Adams, et al., by viewing the gastroesophogeal junction (GEJ) through the endoscope. The fastener is preferably made of polypropylene but can alternatively be made of a biocompatible material and can be a T-fastener. The fastener is inserted through a hypotube which first penetrates tissue of both the esophageal and gastric walls and then the T-fastener is inserted so the distal end of the T-fastener engages the gastric wall as the hypotube is retracted, and then the hypotube is further retracted into the sheath of a fastener delivery device. The proximal end of the T-fastener is pulled out of the hypotube by the tension exerted from the distal end of the T-fastener and then the proximal end seats against the inner wall of the esophagus, thereby holding the gastric wall and the esophagus together. One of the T-fasteners has a simple T-bar at each end, referred to herein as a double tilt tag fastener since the T-bars are normally biased in a T-configuration with respect to the longer joining portion but can be maintained in a reduced diameter configuration, with the T-bars “tilted” or compressed toward the longer joining portion, while inserted and maintained in a hypertube such as a hypodermic needle delivery device. Such fasteners are advanced through the hypertube until the distal portion exits the distal end of the hypertube delivery device and then regains its normally radially expanded position. In Adams, the T-bars when placed on both sides of the intussusception are visible on both sides with conventional endoscopes as one T-bar is on the esophageal side and the other one on the gastric fundal side. No echoendoscope is necessary and none is described. The Adams T-bars are not used to hold a device attached to the mucosa.

A system for suturing, tissue fixation, and transgastric penetration to facilitate surgery on the wall of the GI tract and adjacent hollow organs under Endoscopic Ultrasound (EUS) control was described by Fritscher-Ravens, et al., in Gastrointestinal Endoscopy, vol. 56, No. 5, 2002, pgs. 737 to 742. In this paper, one tilt-tag is placed under EUS control through the mucosa. A free piece of thread as described in Figs. A-E on page 738 comes out of the tilt tag and from the tip of the needle that is modified in order to let the thread come out of the needle. The free thread has then to be attached using either suturing systems or pledgets or tying knots that are not easily done or used through a flexible endoscope. Furthermore, the Fritscher-Ravens, et al., system was not described for use for securing medical devices, for example a GARD, to the gastrointestinal tract or any other organ.

A rigid rather than flexible laparoscopic surgery appliance for installing temporary plastic resorbable tilt tag fasteners, for example for fixing parietal and visceral reinforcements, is described by Bailly, et al., in U.S. Pat. No. 6,779,701. Bailly, et al., teach binding nets for treatment of inguinal hernias. A plunger for forcing the catching bars to pivot is disclosed.

A self-securing suture wire with a T-shaped toggle designed for insertion into a bodily structure, tissue, or organ, delivered by a slotted needle, is disclosed by Levinson, et al., in U.S. Pat. No. 6,596,014. The toggle end portion can be made of nitinol, stainless steel, or biocompatible material. The suture is designed to be placed in a blood vessel from the outside of the vessel.

A T-bar fastener with a sharp end or point on the bar-like head portion, or T, so that the sharp end or point embeds itself in body tissue to securely anchor the T-bar head is disclosed by Richards, et al., in U.S. Pat. No. 4,669,473. A tool receives a fastener with the bar-like head positioned inside a bore of a sheath and the filament portion of the fastener extending out through a slot in the tool bore, the head making a sliding fit in the sheath.

It is an object of the present invention to provide a method and apparatus to effectively secure medical devices to tissues or organs. It is a further object to provide such a method and apparatus for permanently securing a GARD valve to a hiatus hernia or inner mucosa of a gastro-intestinal tract of a patient suffering from GERD.

### SUMMARY OF THE INVENTION

These objects, and others which will become apparent from the following disclosure, are achieved by the present invention which in one aspect comprises a novel double tilt tag staple constructed of a nickel-titanium alloy such as nitinol.

Another aspect of the invention is a surgical method comprising attaching medical device to the inner mucosa of a gastro-intestinal tract of a patient with a double tilt-tag staple using ultrasound real time visualization and a flexible endoscope.

A further aspect of the invention is a special flexible endoscope apparatus and associated endoscopic ultrasound (EUS) tools for securing a medical device to a person's tissue or organ.

In the case of the GARD, the device is preferably inserted though the mouth and the esophagus, and when located at the intersection of the esophagus and stomach, a ring portion is stapled to a hiatus hernia with double tilt tag staples using ultrasound real time visualization and a flexible endoscope, which together facilitate precise placement and stapling.

The tilt-tag staple comprises an elongated joining member having a first end and a second end, a first tilt-tag joined to the first end of the joining member and a second tilt-tag joined to the second end of the joining member, each of the first and second tilt-tags normally aligned perpendicularly to the joining member, the tilt-tags bendable under resistance into a position substantially parallel to the joining member and returning to the normal position when resistance is removed. The tilt-tag staple is preferably constructed of nickel-titanium alloy, and tilt-tag staples of such material are novel and advantageous for several reasons. For temporary applications where temporary tilt-tag staples are needed, the tilt-tag staples can be constructed of biodegradable material, as long as the material is elastic enough to return to normal position after bending the tags to a substantially parallel position during the insertion steps. Such temporary applications include stapling to the esophagus or gastro-intestinal tract of captors monitoring pressure, pH and/or temperature or any other physical parameter. The biodegradable tilt-tags can be made of absorbable materials such as lactide, glycolide, or e-caprolactone for such temporary placement.

For permanent applications such as with a GARD, the joining material can be made in implant grade steel or preferably nitinol, surgical thread, or implant grade polymer such as nylon. The tilt-tags at each end of the joining member can also be made of steel, nitinol, implant-grade polymers such as nylon or any other biocompatible implantable material. In a preferred embodiment, the tilt-tags and joining segment are all made of a non-absorbable material, preferably in nitinol so as to have the flexibility for the tilt-tag to resume an angle from a parallel position when released.

Nitinol in its austenitic configuration has good elasticity properties which tend to restore the original right angle position between the joining member and the tags after release from their parallel insertion position. When the nitinol temperature is cooled as when spraying ice-water directly through the working channel of the endoscopic with ice water, the austenitic harder hyperelastic configuration can be changed into a martensitic much softer configuration. This new configuration makes it much less traumatic to pull on the tilt-tag (note see FIG. 4) to pull out the tilt-tag and remove the GARD allowing replacement.

### BRIEF DESCRIPTION OF THE DRAWINGS

FIG. 1 illustrates a perspective view of a double tilt tag staple according to the invention.

FIG. 2 illustrates a perspective view of a slit needle loaded with one tilt-tag staple in a flexible tube according to the invention.

FIG. 3 illustrates a partial cutaway view of a GARD within an inner mucosa with one tilt-tag staple of the invention inserted through both the GARD and the inner mucosa.

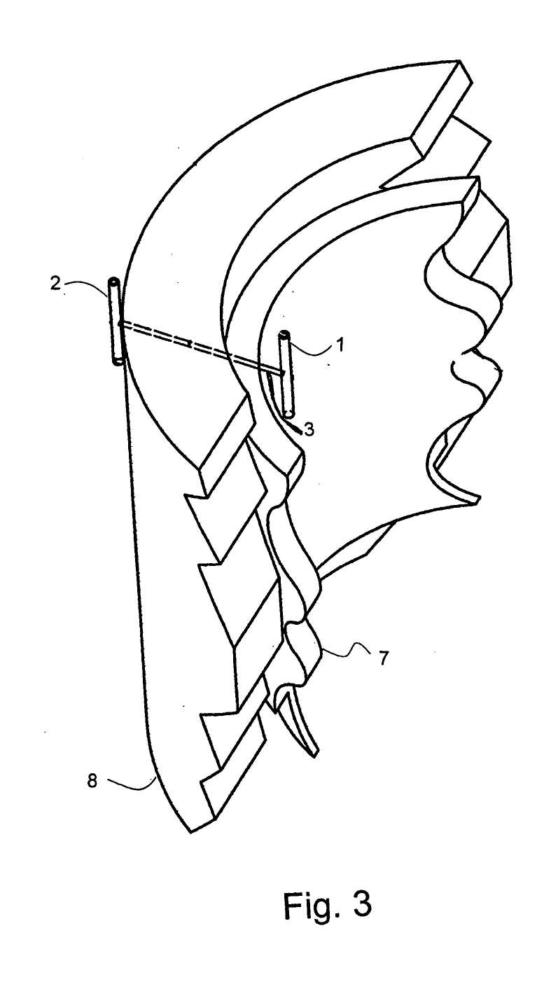

FIG. 4 illustrates the GARD stapled to the inner mucosa of FIG. 3, with the GARD being pulled out toward the inside in an uninstalling operation.

FIG. 5 illustrates a novel two-lumen needle used to insert tilt-tag staples of the invention in some embodiments of the invention.

FIG. 6 illustrates another embodiment of the tilt-tag staple wherein an electrical lead is attached to the flexible joining member.

### DETAILED DESCRIPTION

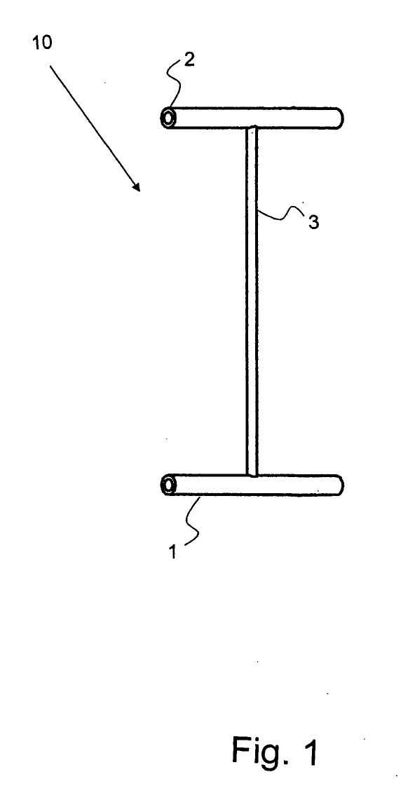

Referring first to FIG. 1, an example of a tilt-tag staple 10 of the invention is shown wherein a first tilt tag 1 and a second tilt tag 2 are joined perpendicularly at respective ends of joining member 3. In the illustrated embodiment, the tilt-tag staple 10 is molded in one piece from Nitinol, a commercially available nickel-titanium alloy.

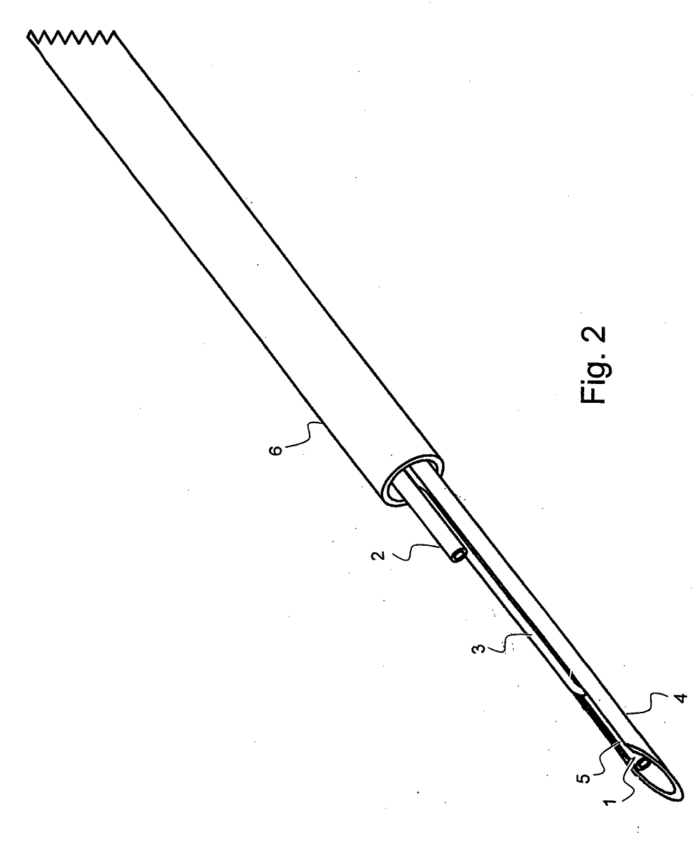

Referring now to FIG. 2, the first tilt tag 1 is inserted in a needle 4 having a slit 5 which is slightly wider than the diameter of the joining member 3 but is less wide than the diameter of the first tilt tag 1 so as to allow the first tilt tag 1 to slide in the channel of the needle 4 and for the joining member 3 to bend in a direction parallel or very close to parallel with the first tilt tag 1. The second tilt tag 2 is bent substantially parallel to the joining member 3 but is outside of the hollow needle channel. The positions of the second tilt tag 2 and the joining member 3 are maintained by the flexible tube 6, and the needle and tilt-tag staple can slide together within the flexible tube 6. The flexible tube 6 acts as a sheath for the needle 4 and tilt-tag staple assembly. The flexible tube 6 is preferably a polyimide tube, for example those sold by Microlumen, Inc. under the trademark Microlumen.

The flexible plastic tube 6 with the needle 4 and the second tilt-tag 2 is passed in the working channel of en endoscope, preferably an echoendoscope. Once the plastic catheter 6 has exited from the working channel at the tip of the endoscope, the needle 4 and first tilt-tag 1 are slid out of the catheter and the needle 4 with an echogenic tip, under ultrasound control, will be pushed into the mucosa 8, through the ring 7 of the GARD. Once the tip of the needle 4, visible under ultrasound, has reached the other side of the wall, in this case the hiatus hernia, the first tilt-tag 1 is pushed out of the needle 4 that acts as a trocar with a pusher (not shown). This procedure is done under ultrasound control as to not cause lesions of nearby organs such as the aorta or vena cava. The needle 4 is then pulled back and the second tilt-tag 2, that was not in the needle 4, will be thus within the lumen 8 of the hernia. The double tilt-tag staple 10 will thus hold the ring 7 of the GARD in place and act as a tilt-tag staple 10 placed through a flexible endoscope. Several tilt-tag staples 10 will be placed around the perimeter of the ring 7 and can be placed in one catheter 6.

The tilt tags 1, 2 are illustrated in nitinol, 0.5 mm in diameter and 6 mm in length, but the diameter of the tilt-tags can vary between 0.1 mm and 3.0 mm or more. The length of the extremity segments of the tilt-tags 1, 2 can vary between 2 mm and 50 mm or more. The length of the nitinol thread flexible joining member 3 is 10 mm long in the illustrated embodiment, but of course can be of other lengths as appropriate to the application, for example between 1 mm and 50 mm or more. Ideally, the first 1 and second 2 tilt-tags and the joining member 3 are made in a single piece of nitinol so as to avoid welding, setting or gluing, which can weaken the structure of the tag and cause rupture. Manufacturing the nitinol tilt-tag in one piece without welding also contributes in allowing conformational change of the nitinol from the hyperlastic austenitic phase use for placement of the tilt-tag to the martensitic, softer phase useful for a less traumatic removal.

In this way, without tying complicated knots or using any pledgets or other devices to attach the free piece of thread, a double tilt-tag can be placed through the mucosa and hold any device in place that needs to be held either for a long period of time such as the GARD that is placed for years, or shorter periods of time such as pressure, pH monitoring devices that can be placed with absorbable tilt-tags for a few weeks. When used with absorbable tilt-tags, the monitoring devices will fall in the lumen of the esophagus, stomach or bowel once the tilt-tags and joining segment dissolve and will be expelled naturally.

In thicker organs, such as the stomach, it is possible to put the tilt-tag with a rigid trocar through the abdominal wall, through the outer layers of the stomach wall within the wall of the stomach, preferably in the muscularis propria layer, particularly to place electrical leads in the stomach wall that are then fixed to an electrical pace-maker, in particular for the treatment of gastroparesis as in the Enterra product sold by Medtronic Inc. for Gastric Electrical Stimulation or for the treatment of obesity as in the Product developed by Transneuronix. The extremity of the lead, for example the Medtronic 4351 Gastric lead is modified.

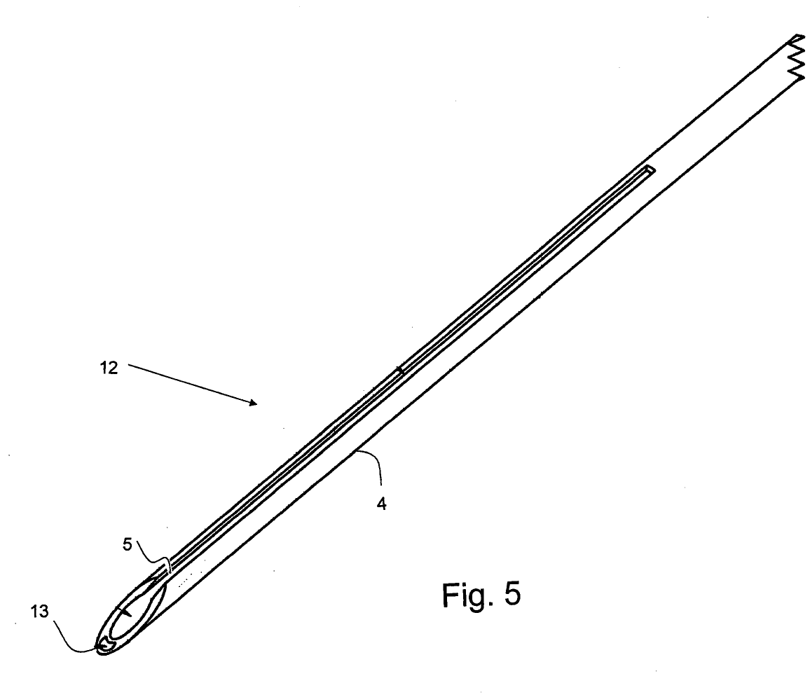

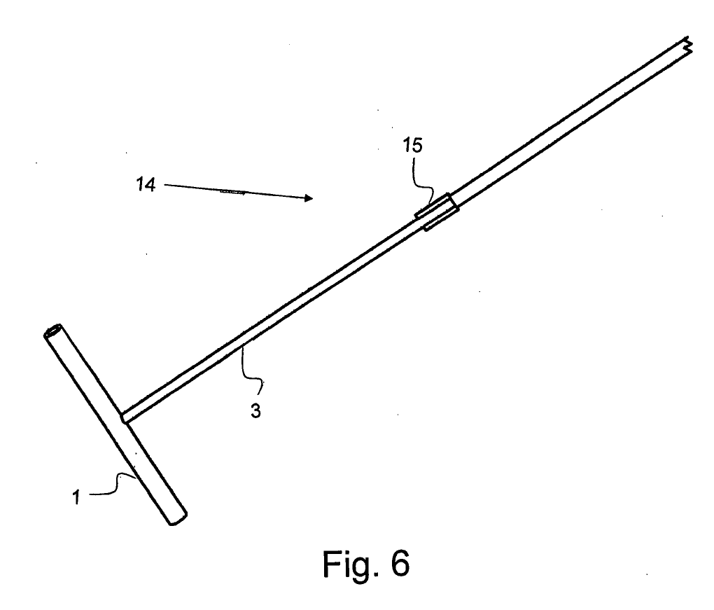

Referring to FIG. 6, a electrical lead terminated tilt-tag staple 14 is illustrated wherein the conventional needle and thread used to fix the tip or the lead with the electrode for placement under surgery is replaced by a nitinol tilt-tag 1 and electrode 15, joined by flexible joining member 3. In order to place the lead under local anaesthesia (as opposed to general anaesthesia which is needed for conventional placements), a double lumen catheter as illustrated in FIG. 5 is used. The second lumen 5 carries the tilt-tag 1 with the electrode of the pace-maker lead 15 placed in a slit needle 4 and the second lumen 13 is used to inject a saline solution. First saline is injected, once the double lumen needle 12 has penetrated in the wall of the stomach and created a saline-filled cavity that appears black on ultrasound in the wall of the stomach, while the tissues are light grey. Then the tilt-tag staple 14 with the electrical lead 15 is fired in that cavity. The fact that the needle 12 used has a double lumen or that two catheters are fused together allows precision placement of the tilt-tag 1 in the wall of the stomach in the muscular layer where the electrode 15 will stimulate the muscle layer.

The double lumen slit needle 12 or fused double catheter approach can be used in an “inside-out” approach as described above, using a flexible double lumen catheter through a flexible echo-endoscope from inside the stomach, but is preferably used in an outside-in approach, using a rigid trocar puncturing through the abdominal wall, then the stomach, placing a lead with an electrode bearing a tilt-tag penetrating from the external layers of the stomach (serosa) into the muscular layer (muscularis) with an ultrasound placed in the stomach such as a radial echoendoscope or a linear (sectorial) endoscope or ultrasound mini-probes made by the Olympus Corp and Microvasive, a Division of Boston Scientific Corp, placed through the working channel of a gastroscope.

This method can be used under local anaesthesia, through the abdominal wall without surgery and general anaesthesia to place a lead in the thickness of the gastric wall, an echoendoscope, guiding the surgeon placing the trocar through the abdominal wall helps him place the lead with electrode into the thickness of the gastric wall. Saline is injected in the wall through one of the lumens to determine the position and create a cavity, the echogenic tip of the needle helping visualize the tip of the needle and the exact depth of penetration. Saline in injected once the tip of the needle has reached the muscularis layer of the gastric wall, then the tilt-tag with the lead is fired in the wall. This technique will allow placement for example of the Enterra device of Medtronic used for treatment of gastroparesis without surgery as well as the lead used by Transneuronix for pacing of the stomach in the treatment of obesity. This method allows placement of electrical leads and their electrode used for gastric pacing, the other end of the lead is attached to a pace-maker for the treatment of gastroparesis (a condition where the stomach contractions are abolished or decreased and can be restored by pacing or for the treatment of morbid obesity as described in Transneuronix's Web Site, currently www.transneuronix.com. Until now, a surgical approach, usually laparoscopic, using general anaesthesia was necessary to place the leads. The placement of the lead 13 with a tilt-tag 1 at its end through a double lumen trocar will allow proper placement of the lead of the pace-maker within the wall of the stomach without penetrating in the stomach cavity and puncturing the gastric mucosa.

This procedure can be done under local anaesthesia made at the place of penetration of the double-lumen trocar in the abdominal wall.

In some instances, it might be useful nonetheless, to place a tilt-tag 1 with an electrode 15 through the gastric wall from outside using a rigid trocar and place the tilt-tag 1 within the stomach, which is an easier procedure and requires only a standard gastroscope for vision without ultrasound capacity. The electrode 15 is placed behind the tilt tag 1 in the wall of the stomach and the lead comes out of the abdominal wall. The lead 15 is then attached in a usual way to the pace-maker and can be placed in the subcutaneous tissue or carried in a belt. If one wants to remove the lead, the tilt-tag is cut off with a gastroscope using standard endoscopic scissors or thread-cutters made by Olympus Corporation, the tag is removed through the mouth and the lead with electrode are just pulled out of the gastric cavity. This system could be useful for the treatment of obesity with the possibility of removing the electrode, lead and pace-maker after a number of months.

The ultrasound used to control the placement of the tilt-tag 1 in the wall can be placed in the lumen of the gastro-intestinal tract such as an echo-endoscope or with a conventional endoscope placed outside the body on the surface of the skin and the echogenic tip trocar can then be seen penetrating the wall of the abdomen. In this approach, in order to see the anterior wall of the stomach clearly, it is better to have a patient drink a half a quart to a quart of water that allows better visualisation of the anterior gastric wall. When an echoendoscope is used a balloon filled cuff is often used to improve ultrasound visualization.

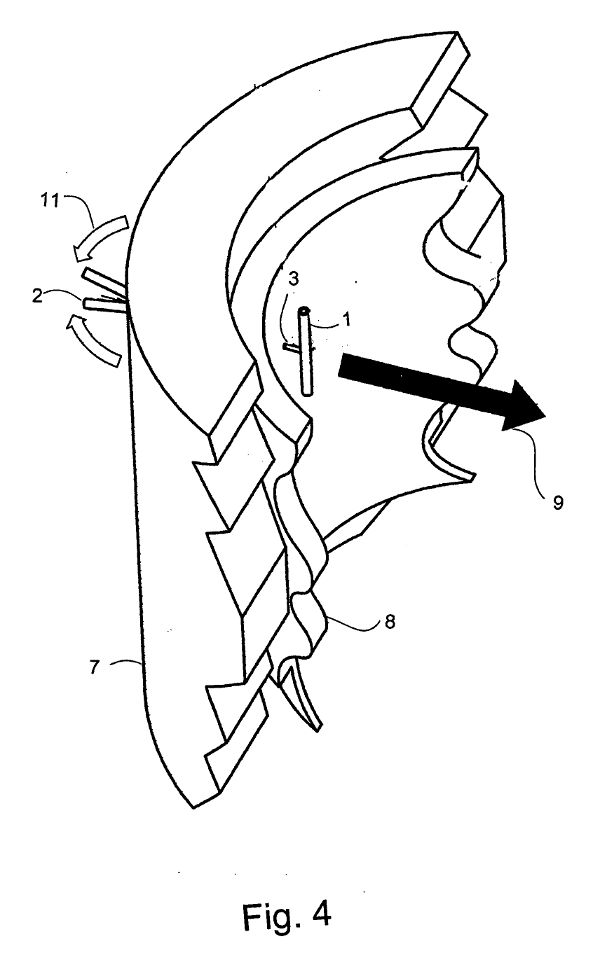

Referring to FIG. 4. in order to remove a device 8 held by a double tilt-tag staple 10, it is possible to cut the flexible segment 3 joining tilt-tags 1 and 2 using a standard Olympus thread cutter. It is also possible, when a polymer such as an implantable polymer is used, to pull the tilt-tag staple 10 with a forceps in direction 9, i.e., toward the inside, and have second tilt-tag 2 outside the lumen fold in the direction indicated by arrows and thus remove the tilt-tag from the wall of the esophagus, stomach or bowel. It is preferable to soften the nitinol tilt-tag prior to pulling the tilt-tag staple through the wall of the esophagus, stomach, or bowel. One method of softening a tilt-tag is to cool it down the with ice water in order to soften the austenitic nitinol into its much softer martensitic configuration, allowing the tilt-tag 2 outside the lumen to fold more easily and avoid tissue lesions when pulled out.

In another embodiment, when a non-absorbable tilt-tag such as a biocompatible nylon is used, it is possible to pull on the tilt-tag with an endoscopic biopsy forceps and pull the tilt-tag placed outside of the lumen by folding the tilt-tag like an arrow to pull it back into the lumen. In this way, it is possible to remove a tilt-tag staple 10 completely. However, cooling the nitinol to soften it, as described above, is a preferred step in removing the staple.

While the invention has been described and exemplified in detail herein, various modifications, alternatives, and improvements should become apparent to those skilled in the art without departing from the spirit and scope of the invention.

What is claimed is:

1. An article comprising an elongated joining member having a first end and a second end, a first tilt-tag joined to the first end of the joining member and a second tilt-tag joined to the second end of the joining member, each of the first and second tilt-tags normally aligned perpendicularly to the joining member, the tilt-tags bendable under resistance into a position substantially parallel to the joining member and returning to the normal position when resistance is removed, the article constructed of nickel-titanium alloy.

2. A surgical method comprising attaching medical device to the inner mucosa of a gastro-intestinal tract of a patient with a double tilt-tag staple using ultrasound real time visualization and a flexible endoscope.

3. The method of claim 2 wherein the endoscope has a working channel and a pusher in the working channel, the double tilt-tag staple comprising an elongated joining member having a first end and a second end, a first tilt-tag joined to the first end of the joining member and a second tilt-tag joined to the second end of the joining member, each of the first and second tilt-tags normally aligned perpendicularly to the joining member, the tilt-tags bendable under resistance into a position parallel to the joining member and returning to the normal position when resistance is removed, the method comprising placing the joining member and the first tilt-tag of a first tilt-tag staple in the channel of a slit needle, placing the slit needle in a flexible tube so that the second tilt-tag is outside the slit needle and within the flexible tube, the tilt-tags maintained in a stressed position substantially parallel to the joining member, placing the flexible tube containing the tilt-tag staple in the working channel of the flexible endoscope, placing the flexible endoscope in the patient's gastro-intestinal tract and through the medical device, directing the needle into and through the inner mucosa portion of the tract, using the pusher to release the first tilt-tag outside of the tract, and pulling the needle back so that the second tilt-tag is released within the tract, thereby stapling the device to the tract.

4. The method of claim 3 wherein comprising placing a plurality of double tilt-tag staples within the needle and stapling the device to the tract at a plurality of locations.

5. The method of claim 2 wherein the device is a gastrointestinal anti-reflux device (GARD) having a collapsible ring, the method comprising stapling the ring in a hiatus hernia at the juncture of the patient's esophagus and stomach at a plurality of locations.

6. The method of claim 2 wherein the tilt-tag staple is constructed of a nickel-titanium alloy, steel, and/or nylon.

7. The method of claim 2 wherein the tilt-tags are constructed of absorbable material and the device is a sensor designed for temporary implantation in the patient.

8. The method of claim 2 wherein the needle comprises a first lumen and a second lumen, using the first lumen for injection of saline and using the second lumen for holding and releasing the tilt-tag.

9. The method of claim 2 wherein the device is a pacemaker and the tilt-tag staples are used for placement of pace-maker leads and electrodes with a rigid trocar through the abdominal wall into the stomach or from outside the stomach inward.

10. A method of removing a tilt-tag staple having a nitinol tilt-tag, the staple previously implanted in a patient's esophagus, stomach, or bowel, comprising a step of cooling the nitinol tilt-tag to soften the tilt-tag.
